(19) United States
(12) Patent Application Publication
(10) Pub. No.: US 2025/0160693 A1
(43) Pub. Date: May 22, 2025

(54) METHODS AND SYSTEMS FOR PROCESSING NON-INVASIVE BLOOD MONITOR DATA

(71) Applicant: JRE Star Investment Holdings, LLC, Farmington, UT (US)

(72) Inventors: Tyler Jamison Jarvis, Provo, UT (US); Andrew Isaac Williams, Provo, UT (US)

(21) Appl. No.: 18/955,864

(22) Filed: Nov. 21, 2024

Related U.S. Application Data
(60) Provisional application No. 63/601,335, filed on Nov. 21, 2023.

(51) Int. Cl. A61B 5/145; A61B 5/00; A61B 5/024

(52) U.S. Cl. CPC A61B 5/14532; A61B 5/024; A61B 5/7246; A61B 5/725; A61B 5/7257; A61B 5/7275

(57) ABSTRACT

Systems and methods for processing signals from a non-invasive blood analyte measurement system. In some such methods, signal data may be received from a non-invasive blood monitor. The signal data may comprise frequency data corresponding to a heart rate pulse of a user of the non-invasive blood monitor. A Fourier transform of one or more sections of the signal data may be performed. A subset of modes from the Fourier transform may be selected and data from the Fourier transform may be used to initialize an adaptive, linear signal model. Kalman filtering may then be applied to the linear signal model to update the linear signal model by propagating the subset of modes and processing a dynamic projection of the signal data onto the subset of modes.

### CROSS-REFERENCE TO RELATED APPLICATIONS

This application claims the benefit under 35 U.S.C. § 119 (e) of U.S. Provisional Patent Application No. 63/601,335, which was filed Nov. 21, 2023, and titled “Fourier-Kalman State (FoKS): A Flexible Framework for Modeling and Prediction of Periodic Systems,” which is hereby incorporated herein by reference in its entirety.

### SUMMARY

Embodiments of apparatus and methods are disclosed herein that relate to processing of data for time-varying systems, such as processing data from a non-invasive blood monitoring system.

Kalman filtering has been established as a powerful tool for state estimation given noisy data. This filtering technique emerges as a viable option for estimation and smoothing when the data-generating process has stationary dynamics. However, in the case of time-varying processes, the standard Kalman update equations cannot adapt to these changes in the generation process and thus often perform poorly in such circumstances.

Disclosed herein are therefore various methods and systems for continuous basis projection Kalman filtering, which allows for a time-varying loading to be applied to a sparse set of basis functions, thereby allowing for coherent structure to be represented in the signal dynamically. The efficacy of this approach is established for the real-time processing of EEG signals, which may lead to improvements in neurological signal processing and information extraction. This approach may also be used to process other data, such as data used in non-invasive blood monitoring and/or analysis, such as in glucose monitoring systems for managing diabetes.

In an example of a method for processing signals, such as signals from a non-invasive blood analyte measurement system, the method may comprise receiving signal data, such as receiving signal data from a non-invasive blood monitor or an EEG. In some cases, the signal data may comprise frequency data corresponding to a biological signal, such as a heart rate pulse of a user of the non-invasive blood monitor. The method may further comprise performing a Fourier transform of one or more sections of the signal data. A subset of modes from the Fourier transform may then be selected. Data from the Fourier transform may be used to initialize an adaptive, linear signal model. Kalman filtering may then be applied to the linear signal model to update the linear signal model by propagating the subset of modes and processing a dynamic projection of the signal data onto the subset of modes.

Some implementations may further comprise using the linear signal model to predict movements in a blood analyte concentration and/or to predict a blood analyte concentration. In some cases, the blood analyte may comprise glucose.

In some implementations, the step of applying Kalman filtering to the linear signal model to update the linear signal model may comprise adaptively updating the linear signal model to track signal dynamics using Kalman updates, updating an estimate of the linear signal model, and/or comparing the updated estimate of the linear signal model to new signal data received, such as new signal data received from the non-invasive blood monitor.

Some implementations may further comprise identifying one or more parameters associated with a shape of the heart rate pulse. In some cases, this may be accomplished using a machine learning model. Some such implementations may further comprise using the one or more parameters to predict a blood analyte condition.

In an example of a method for identifying one or more features of a shape of a pulse signal, such as a heart rate pulse, using a non-invasive monitor, the method may comprise receiving signal data from a non-invasive blood monitor. The signal data may comprise frequency data corresponding to a heart rate pulse and/or other physiological and/or pulsatile signal of a user of the non-invasive blood monitor. One or more features of the signal data may be extracted, in some cases using a dynamic projection of the signal data onto Fourier modes. One or more extracted features may then be correlated from the signal data with a blood analyte condition, such as blood glucose levels. A blood analyte condition may then be predicted and/or estimated using the one or more extracted features.

In some implementations, the blood analyte condition may comprise a concentration of the blood analyte. Alternatively, or additionally, the blood analyte condition may comprise a trend over time of the blood analyte.

In some implementations, the step of extracting one or more features of the signal data may comprise use of a Kalman filter.

In an example of a method for predicting a condition, such as a blood analyte condition, using data from a monitor, such as a non-invasive blood monitor, the method may comprise receiving signal data, such as receiving signal data from a non-invasive blood monitor. In some cases, the signal data may comprise frequency data, such as frequency data corresponding to a heart rate pulse or other pulsatile signal of a user of the non-invasive blood monitor. One or more parameters associated with a shape of the heart rate pulse or other pulsatile signal may be identified. In some cases, such parameters may be identified using machine learning techniques. The one or more parameters may then be used to predict a condition, such as a blood analyte condition, from the signal data.

In some implementations, the blood analyte condition may comprise a trend over time in a concentration of the blood analyte. Alternatively, or additionally, the blood analyte condition may comprise a concentration of the blood analyte.

In some implementations, the step of identifying one or more parameters comprises performing a Fourier transform of the signal data.

Some implementations may further comprise applying Kalman filtering to data resulting from the Fourier transform. In some such cases, the step of applying Kalman filtering to data resulting from the Fourier transform may comprise using data from the Fourier transform to initialize an adaptive, linear signal model, and/or applying Kalman filtering to the linear signal model to update the linear signal model.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a flowchart illustrating an example of a method for processing physiological data using a combination of Fourier and Kalman filtering techniques;

FIG. 2 is a schematic diagram of a system for processing pulsatile signal data; and

FIG. 3 is another flowchart illustrating another example of a method for processing physiological data according to other implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

Various techniques are disclosed herein that allow for modeling of periodic signals, such as pulsatile signal modeling, which may allow for estimating, predicting, correlating, and/or tracking a condition, such as a blood analyte condition. In some embodiments and related implementations, this may be accomplished by combining Fourier transforms and/or analysis of the data with Kalman filtering.

Given an underlying data generation model expressed as a linear dynamical system, the Kalman filter can reject noise and produce good estimates of the true state of the system by leveraging the structure of the state transition function. This structure allows the noise rejection properties of the Kalman filter to be better than models that encode less structure, such as ARMA, ARIMA, and LSTM, in many applications. However, if the underlying dynamics change, the Kalman filter cannot adapt to these changes while maintaining good performance. Related work includes switching Kalman filters, which establish a convex combination of Kalman filters to describe systems that may not be described by a single dynamics function. This disclosure provides a more nuanced and adaptive approach suitable for online estimation of the state of a time-varying system, such as a glucose monitoring system or any other blood analyte monitoring system.

A general theory is therefore provided herein for basis projection Kalman filtering, which allows for a dynamic representation of the dynamics, broadening the scope of Kalman filtering to signals not generated by a stationary system. Domains where such signals occur frequently include physiological signals, such as heartbeats, respiration, and other biological rhythms, seismic activity, and economic indicators, to name a few.

For embodiments and implementations in which the aim is to project an observed signal onto a set of basis functions, we first consider the canonical basis for compactly supported continuously differentiable functions C_{c}¹, the Fourier basis. Then given some periodic signal f(t), we may write:

$$f ( t ) = {\sum }_{k = - ∞}^{∞} {c}_{k} {e}^{i 2 π kt} . \qquad ( 1 )$$

In this equation, cₖ are the Fourier coefficients. In order to represent this basis using a Kalman system, it may be helpful to analyze the Fourier modes more directly. Note that because a sparse representation may be desired, the input signal may preferably be periodic to allow for good reconstruction with minimal basis functions. If the signal we wish to model is not periodic, other dynamics bases, may be used, as discussed below. However, for certain preferred applications, the Fourier basis may be used, as it is a flexible and useful approach for many signal models.

In order to model the Fourier basis with a Kalman filter, the dynamics may be represented with a linear differential equation. Thus, we seek a differential equation with solution cₖe^{iπkt}, so any signal may be decomposed into a finite sum of these dynamics functions. These complex exponentials are known to be the solutions to the undamped oscillator system, known as Hooke's law, given by:

$$\frac{{\partial }^{2} x}{\partial {t}^{2}} = - {c}^{2} x .$$

This equation describes harmonic motion, and its general solution can be expressed as:

x(t)=e^{ict}

Note that these solutions correspond precisely to the Fourier basis in the first equation above if we set c=k. To model this in a dynamic system conducive to filtering, we consider the first-order system of differential equations describing Hooke's law:

$$( \begin{matrix}{\dot{x}}_{1} \\ {\dot{x}}_{2}\end{matrix} ) = ( \begin{matrix}0 & 1 \\ - {c}^{2} & 0\end{matrix} ) ( \begin{matrix}{x}_{1} \\ {x}_{2}\end{matrix} ) .$$

The solution to this system's evolution over time can be found using the matrix exponential. The fundamental matrix solution, denoted by \hat{Φ}, is given by \hat{Φ}=exp(D), where

$$D = ( \begin{matrix}0 & 1 \\ - {c}^{2} & 0\end{matrix} ) .$$

This matrix D encapsulates the dynamics of the system.

For practical implementation in a Kalman Filter, where data arrives in discrete time steps, we can modify this to: Φ=exp(D·h), where h is the size of the time step. This allows us to express a single complex exponential as a linear system, suitable for incorporation into the Kalman filtering framework.

For development of signal modeling techniques for machine representation, because any sampled signal will be operated on discretely, we can use the Discrete Fourier Transform as the orthonormal expression for f(t). Then we have for f represented discretely as f(n):

$$f ( n ) = \frac{1}{N} {\sum }_{k = 0}^{N - 1} {c}_{k} {e}^{\frac{i 2 π}{N} kn}$$

with n∈ [symbol] . This expression leads us to a relaxation of the standard definition of the Fourier transform, with a finite number of basis elements used for representation. If we wish our representation to be agnostic to the size of the dataset, we may instead represent the signal as:

$$f ( n ) = {\sum }_{k = 0}^{N - 1} {c}_{k} {e}^{i 2 π {w}_{k} t}$$

In this equation, we can relax the wₖ, and allow wₖ∈ [symbol] . Then, as long as t is sampled on the grid defined by the signal sample frequency fs, where we have the step h satisfying h=1/fs, the representation above is well defined. Note here the wₖ do not have a fixed bin width as they would in the corresponding DFT expression, as the signal expression does not depend on the number of samples used to initialize the expression.

Then, given this expression, we can define:

$${Φ}_{k} = exp ( \begin{matrix}0 & h \\ - {hw}_{k}^{2} & 0\end{matrix} )$$

Then, for a given set of frequencies {w₁ . . . wₖ}, we have that the linear system defining the time evolution of each basis frequency is given by:

$$( \begin{matrix}{x}_{{w}_{1}}^{t + h} \\ {\dot{x}}_{{w}_{1}}^{t + h} \\ ⋮ \\ {x}_{{w}_{k}}^{t + h} \\ {\dot{x}}_{{w}_{k}}^{t + h}\end{matrix} ) = ( \begin{matrix}{Φ}_{1} & \begin{matrix}0 & 0 \\ 0 & 0\end{matrix} & … \\ \begin{matrix}0 & 0 \\ 0 & 0\end{matrix} & ⋱ & ⋮ \\ ⋮ & … & {Φ}_{k}\end{matrix} ) \cdot ( \begin{matrix}{x}_{{w}_{1}}^{t} \\ {\dot{x}}_{{w}_{1}}^{t} \\ ⋮ \\ {x}_{{w}_{k}}^{t} \\ {\dot{x}}_{{w}_{k}}^{t}\end{matrix} )$$

Where x_{w}_{ₖ} is the position of each wave in time, and x_{w}_{ₖ} is the derivative of position, which gives the system of first-order coupled ODEs describing the time evolution of each complex exponential as in the first-order system of differential equations describing Hooke's law above.

Then we have that f(t) is given by the sum of the value of complex exponentials times the Fourier coefficients cₖ, and so if the equation above describes the time evolution of the e^{i2πw}^{ₖ}^{t}, then f(t) is given by the coefficients (alternating with zero) dotted with the frequency state vector. Thus, we have:

$$f ( t ) = ( {c}_{1} 0 … {c}_{k} 0 ) \cdot {( {x}_{{w}_{1}}^{t} {\dot{x}}_{{w}_{1}}^{t} … {x}_{{w}_{k}}^{t} \dot{x} {m}_{k}^{t} )}^{T}$$

Then f(t) obeys the following update equations:

$$\begin{matrix}{x}^{t + h} = A \cdot {x}^{t} + {ε}_{K} \\ f ( t + h ) = C \cdot {x}^{t + h} + {ε}_{R}\end{matrix}$$

Where ε_{K} and ε_{R} are Gaussian noise with mean zero and covariance K,R, respectively. Note that if we wish to express a multidimensional signal f(t) with m dimensions, we can simply expand the C and R matrices accordingly:

$$C = ( \begin{matrix}{c}_{11} & … & {c}_{1 j} \\ 0 & 0 & 0 \\ & ⋮ & \\ {c}_{1 k} & … & {c}_{km} \\ 0 & 0 & 0\end{matrix} ) R = ( \begin{matrix}{σ}_{{obs}_{1}}^{2} & 0 & … \\ 0 & {σ}_{{obs}_{2}}^{2} & … \\ ⋮ & ⋱ & ⋮ \\ 0 & … & {σ}_{{obs}_{m}}^{2}\end{matrix} )$$

Where each dimension of f(t) has a representation, as indicated in the equation representing the signal above, where each of the frequencies wₖ must be the same for each dimension, but the cₖ can change between dimensions.

Finally, to allow for the expression of signals with time-varying generation processes, we can allow the cₖ to be included in the state x^{t}, giving rise to the simplest incarnation of dynamic projection filtering. This dynamic Fourier projection is a powerful technique for filtering and inference for signals with consistent frequency content but time-varying expressions of the underlying frequencies.

Thus, expanding the state variable x^{t}, we arrive at the following Kalman system in the case of a single signal dimension:

$${\tilde{x}}^{t} = ( \begin{matrix}{x}_{{w}_{1}}^{t} \\ {\dot{x}}_{{w}_{1}}^{t} \\ ⋮ \\ {c}_{1} \\ ⋮ \\ {c}_{k}\end{matrix} ) \tilde{A} = ( \begin{matrix}A & 0 \\ 0 & I\end{matrix} ) \tilde{C} = ( \begin{matrix}1 & 0 & 0 & … \\ 0 & 0 & 1 & … \\ ⋮ & & ⋱ & ⋮ \\ 0 & … & 1 & 0\end{matrix} ) \cdot ( \begin{matrix}0 \\ I\end{matrix} ) \tilde{K} = ( \begin{matrix}K & 0 \\ 0 & {I}_{ℽ}\end{matrix} )$$

Where l_{γ} is a diagonal matrix, with entries γ that parametrize the uncertainty of the coefficients cₖ. Note here the system update equations are now given by:

$$\begin{matrix}\begin{matrix}{\tilde{x}}^{t + h} & = \tilde{A} \cdot {\tilde{x}}^{t} + {ε}_{\tilde{K}}\end{matrix} \\ f ( t + h ) = {( {\tilde{x}}^{t + h} )}^{T} \cdot \tilde{C} \cdot {\tilde{x}}^{t + h} + {ε}_{R}\end{matrix}$$

Here, the observation function is no longer linear but a quadratic form. Thus, rather than the standard Kalman filter, we may use the quadratic extended Kalman filter (QEKF), which has good convergence properties. Also, note that this method generalizes easily to multiple observation dimensions simply by including more coefficients in the state. However, the observation matrix no longer works out as nicely, so we do not write the corresponding \tilde{C} for this case. In any case, when implementing this algorithm computationally, we would not take the matrix product with a sparse matrix like \tilde{C}; we would simply multiply the x_{w}_{ₖ} with the corresponding cₖ_{₁} . . . cₖ_{ₘ} coefficients in order to produce the output f(t)₁ . . . f(t)ₘ. \tilde{C} is a tool we use for illustration and numerical stability analysis purposes.

Then, to initialize a basis system, we can calculate the power spectrum of a section of the signal and initialize the wₖ and cₖ according to the k largest constituents of the spectrum, \hat{f}(w). If the signal is periodic, we may estimate the \hat{f}(ω) using the MUSIC algorithm (MUltiple SIgnal Classification), which often works well when data have significant sinusoidal constituents. In other cases, the modified covariance method may be useful for spectrum estimation.

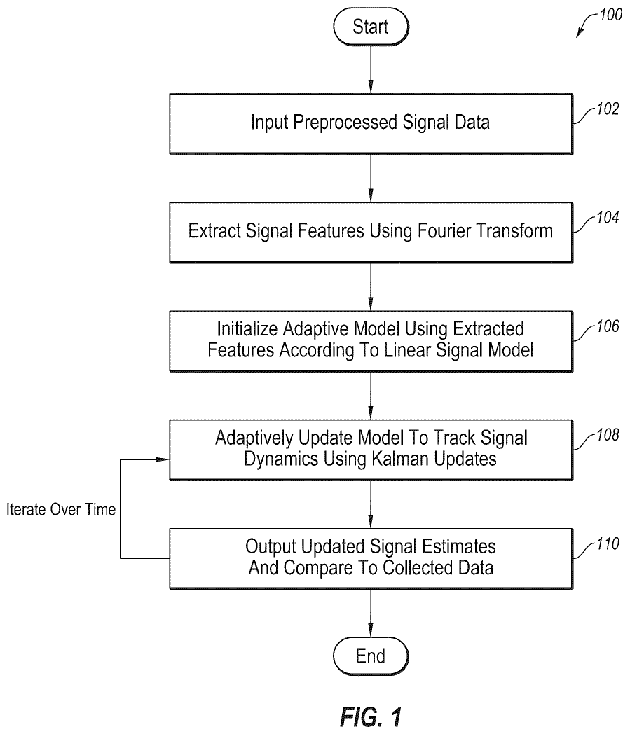

These methods may be used to model periodic signals and estimate and/or predict conditions, such as blood analyte conditions using non-invasive blood monitors. FIG. 1 is a flowchart illustrating a method 100 for processing data, such as non-invasive blood monitor data in some implementations. At step 102, signal data may be received and/or input. In some cases, the signal data may be received from a blood monitor, such as a non-invasive blood monitor. Examples of such monitors can be found in U.S. Pat. No. 11,903,686 titled “Systems, Apparatuses, and Methods for Optimizing a Physiological Measurement Taken From a Subject,” the entire contents of which are hereby incorporated herein by reference in its entirety.

In some cases, the signal data received at step 102 may be preprocessed. For example, in some cases, preprocessing may include one or more smoothers, high-pass, low-pass, and/or band-pass filters. In some such cases, or in some alternative cases, preprocessing in step 102 may comprise averaging of signals from several adjacent wavelengths and/or power spectrum analyses.

Once the signal data, whether preprocessed or raw, has been received, signal features from the data may be extracted. In preferred implementations, these features may be extracted using a Fourier Transform and/or analysis. In some cases, features such as features associated with a shape of a periodic and/or pulsatile signal, such as features associated with a heart-beat signal from a non-invasive monitor, may be extracted. For example, in some cases, the relative amplitudes and/or phases of one or more frequencies may be indicative of analyte concentrations or other analyte conditions.

An adaptive model may then be initialized at step 106. In some cases, this model may incorporate the signal features extracted at step 104 and/or may be used to represent the system according to a linear signal model. For example, in some cases, differential equations describing Hooke's law may be used in order to express the data as a linear system suitable for use in connection with a Kalman filtering framework. As described above, Hooke's law can be used to describe the dynamics of the Fourier modes, which may allow for fitting this dynamic representation of the signal into the Kalman filtering framework.

The model may then be updated to track signal dynamics at step 108. The model may be updated using Kalman updates, as described above. This may allow for updating the signal model given the linear representations provided above, which may be done using Kalman update equations.

Following the generation of Kalman updates, method 100 may proceed to step 110, at which point updated signal updates may be generated and/or output. These updates may be compared with data collected from the blood sensor or other data collection system and used to improve the accuracy of the processed data. For example, in some implementations, the updates may be used to generate and/or output estimates to a user, such as estimates of blood glucose, blood oxygen, or another blood analyte, for example.

Alternatively, the model may be used to predict and/or estimate other aspects of the cardiovascular system, such as real-time blood pressure and/or artery diameter. This data may then, in some cases, be used as input for another model that is configured to use such data to estimate and/or predict blood analyte concentrations or other analyte conditions.

As shown in FIG. 1, and as those of ordinary skill in the art will appreciate, method 100 may be iterated over time to enhance the accuracy of the resulting data output. The illustrated method 100 may be implemented in a variety of ways, including using software, firmware, hardware, and/or any combination thereof.

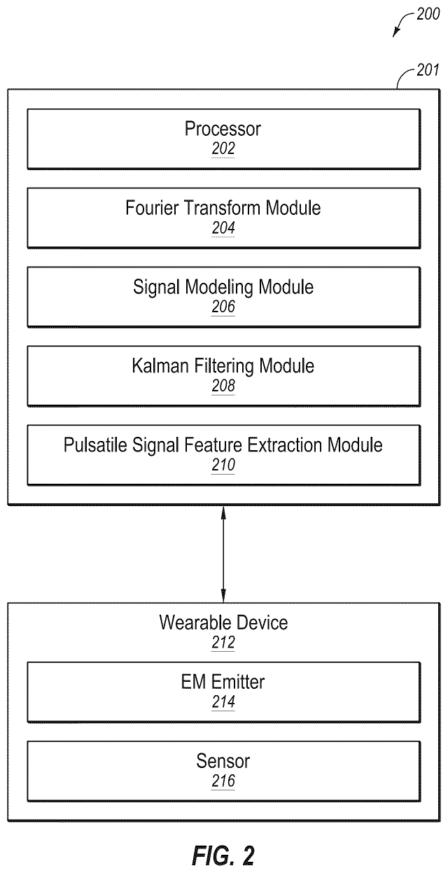

FIG. 2 is a schematic diagram of a system 200 for processing data, such as data from a wearable device 212 used for monitoring health conditions, such as blood analyte conditions, according to some implementations. As shown in this figure, wearable device 212, which may comprise a wrist monitor, comprises one or more electromagnetic emitters 214 and one or more sensors 216. In some embodiments, the wearable device 212 may be positioned on the wrist so that a physiological sensor 216 is positioned adjacent to a particular, targeted physiological feature, such as an ulnar artery or radial artery, for example.

In some embodiments, an electromagnetic emitter 214 may be positioned at a suitable location relative to the sensor 216 so that electromagnetic radiation of a desired wavelength may be emitted from emitter 214, reflected from desired regions within the body, such as along an adjacent to a particular artery in the case of blood monitoring, and then received by sensor 216 for processing according to, for example, the method of FIG. 1 or any of the other methods disclosed herein.

As those of ordinary skill in the art will appreciate, sensor 216 may be configured to convert reflected/incoming radiation into electronic signals, which may be transmitted to and received by processing system 201. For example, in some embodiments, sensor 216 may be configured to measure the intensity of the received radiation over time and transmit this signal to system 201. System 201 may be incorporated onto the wearable device 212 or may be incorporated into another device, such as a mobile application running on a smartphone, a computer, a remote server, or the like.

System 201 may comprise one or more processors 202. Processor(s) 202 may be configured to receive and/or process data from wearable device 212 and/or sensor 216. In addition, processor(s) 202 may be configured to perform various functions, some of which may be part of a particular software module. As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

System 201 may comprise a Fourier Module 204. Fourier Module 204 may be configured to take raw and/or preprocessed data, which preferably includes a periodic feature, and project the signal data onto a set of basis functions. In some embodiments, Fourier Module 204 may be configured to extract one or more features of a periodic aspect of the signal, such as features associated with a shape of a pulsatile signal contained within the data. In some embodiments, certain frequency amplitudes computed by the Fourier Module 204 might be indicative of certain analyte concentrations, but also may be indicative of certain cardiovascular conditions. It may therefore be possible to diagnose, for example, atrial fibrillation or similar conditions by analyzing the shape of the waveform with Fourier analysis.

System 201 may further comprise a Signal Modeling Module 206. In some embodiments, Signal Modeling Module 206 may be configured to model the signal data in a manner conducive to filtering, such as representing the dynamics as a linear system, in some cases using a linear differential equation and/or a differential equation of another kind. Using a mathematical signal model to describe the data allows for the implementation of various algorithms to clean and filter the data, such as using Filtering Module 208 described below.

System 201 may further comprise a Filtering Module 208, which may comprise a Kalman Filtering Module 208 in preferred embodiments. In some embodiments, module 208 may be configured to adaptively update the module created by Signal Modeling Module 206 by using Kalman updates from the incoming data. As those of ordinary skill in the art will appreciate, Filtering Module 208 may be configured to reduce or remove unwanted noise from the signal and/or to enhance the accuracy of the predications/data, such as the extracted analyte conditions and/or other parameters, provided by System 201.

Some embodiments may further comprise a Pulsatile Signal Feature Extraction Module 210. In such embodiments, module 210 may be configured to extract certain features contained within a pulsatile signal received by sensor 216. In embodiments in which system 200 is used to provide blood analyte condition data, such as glucose monitoring data, module 210 may therefore be configured to extract features associated with a shape of a heartbeat signal or other physiological, periodic signal. Such feature(s) may then be used to enhance the accuracy of the data provided to a user by system 200 by, for example, linking certain pulsatile signal shapes with particular blood analyte conditions, such as blood glucose concentrations, other blood analytes, and/or other cardiovascular system parameters, such as blood pressure, arterial elasticity, and the like. Pulsatile Signal Feature Extraction Module 210 may be configured to use, in some cases, a Fourier transform and/or a wavelet transform to extract shape features.

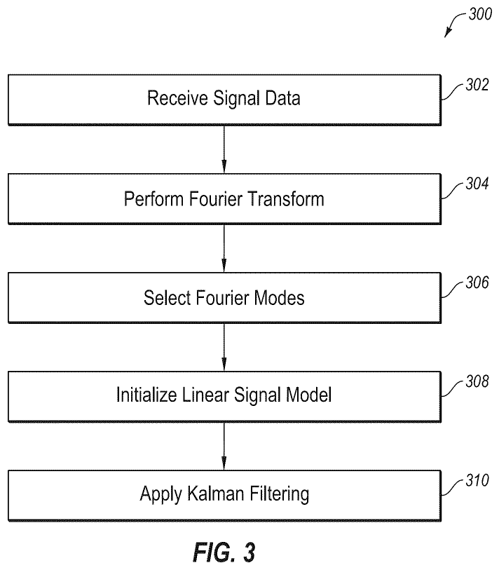

FIG. 3 is another flowchart illustrating another example of a method 300 for processing data, such as blood analyte data from a non-invasive blood monitor, for example. Method 300 begins at 302 at which point data is received. As previously mentioned, such data may be received, for example, from one or more sensors, such as sensors configured to process reflected electromagnetic radiation on a wrist monitor or another suitable wearable device.

The signal data may then be processed at 304 using a Fourier Transform and/or analysis upon the received data. In some embodiments, step 304 may comprise extracting signal features using a Fourier Transform. In some embodiments, the Fourier modes from this transform and/or analysis may then be selected at 306. In some cases, the Fourier modes may be represented using a linear differential equation. As described in greater detail above, in some cases, Fourier modes may be described by a linear differential equation to encode Fourier frequencies into a linear system, which may be conducive to Kalman filtering for subsequent steps.

The Fourier transform, modes, and/or analysis may be used to initialize a linear signal model of the data at 308. In some cases, this may be done using a linear differential equation, in some cases a linear differential equation involving Hooke's law.

Method 300 may then proceed by using Kalman filtering at 310. In some implementations, step 310 may comprise use of dynamic Fourier projections. Step 310 may therefore comprise dynamic signal projections in some cases, but need not in other cases. For example, either a static observation model or a dynamic observation model may be implemented, as desired. Dynamic projection is typically more powerful and expressive, but comes with its own set of compromises. Although static models may be used, it is thought that dynamic projection models may provide deeper insights into the structure of the signal that may be used to predict parameters of interest, such as blood analyte conditions, and/or to describe the cardiovascular system in a way that allows for other models to account for the changes in the cardiovascular system, and thereby more accurately extract parameters of interest.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

In addition, data being tied or rendered together in a database record may be resident in the same memory device, or across several memory devices, and may be linked together in fields of a record in a database across a network. Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMS, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

It will be understood by those having skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A method for processing signals from a non-invasive blood analyte measurement system, the method comprising the steps of:
receiving signal data from a non-invasive blood monitor, wherein the signal data comprises frequency data corresponding to a heart rate pulse of a user of the non-invasive blood monitor;
performing a Fourier transform of one or more sections of the signal data;
selecting a subset of modes from the Fourier transform;
using data from the Fourier transform to initialize an adaptive, linear signal model; and
applying Kalman filtering to the linear signal model to update the linear signal model by propagating the subset of modes and processing a dynamic projection of the signal data onto the subset of modes.

2. The method of claim 1, further comprising using the linear signal model to predict movements in a blood analyte concentration.

3. The method of claim 2, wherein the blood analyte comprises glucose.

4. The method of claim 1, further comprising using the linear signal model to predict a blood analyte concentration.

5. The method of claim 1, wherein the step of applying Kalman filtering to the linear signal model to update the linear signal model comprises:
adaptively updating the linear signal model to track signal dynamics using Kalman updates;
updating an estimate of the linear signal model; and
comparing the updated estimate of the linear signal model to new signal data received from the non-invasive blood monitor.

6. The method of claim 1, further comprising identifying one or more parameters associated with a shape of the heart rate pulse.

7. The method of claim 6, further comprising using the one or more parameters to predict a blood analyte condition.

8. The method of claim 7, wherein the blood analyte condition comprises a blood glucose level.

9. A method for identifying one or more features of a shape of a heart pulse signal using a non-invasive monitor, the method comprising the steps of:
receiving signal data from a non-invasive blood monitor, wherein the signal data comprises frequency data corresponding to a heart rate pulse of a user of the non-invasive blood monitor;
extracting one or more features of the signal data by using a dynamic projection of the signal data onto Fourier modes;
correlating one or more extracted features from the signal data with a blood analyte condition; and
predicting the blood analyte condition using the one or more extracted features.

10. The method of claim 9, wherein the blood analyte condition comprises a concentration of the analyte.

11. The method of claim 9, wherein the blood analyte condition comprises a trend over time of the analyte.

12. The method of claim 9, wherein the blood analyte comprises glucose.

13. The method of claim 9, wherein the step of extracting one or more features of the signal data comprises use of a Kalman filter.

14. A method for predicting a blood analyte condition using data from a non-invasive blood monitor, the method comprising the steps of:
receiving signal data from a non-invasive blood monitor, wherein the signal data comprises frequency data corresponding to a heart rate pulse of a user of the non-invasive blood monitor;
identifying one or more parameters associated with a shape of the heart rate pulse; and
using the one or more parameters to predict a blood analyte condition from the signal data.

15. The method of claim 14, wherein the blood analyte condition comprises a trend over time in a concentration of the blood analyte.

16. The method of claim 14, wherein the blood analyte condition comprises a concentration of the blood analyte.

17. The method of claim 14, wherein the blood analyte comprises blood glucose.

18. The method of claim 14, wherein the step identifying one or more parameters comprises performing a Fourier transform of the signal data.

19. The method of claim 18, further comprising applying Kalman filtering to data resulting from the Fourier transform.

20. The method of claim 19, wherein the step of applying Kalman filtering to data resulting from the Fourier transform comprises:
using data from the Fourier transform to initialize an adaptive, linear signal model; and
applying Kalman filtering to the linear signal model to update the linear signal model.
